(19) United States
(12) Patent Application Publication
(10) Pub. No.: US 2008/0234647 A1
(43) Pub. Date: Sep. 25, 2008

(54) INTEGRATED COMPLIMENTARY PRODUCT

(76) Inventor: Misty Arterburn, McCordsville, IN (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/054,239

(22) Filed: Mar. 24, 2008

Related U.S. Application Data
(60) Provisional application No. 60/896,856, filed on Mar. 23, 2007.

(51) Int. Cl. A61F 13/15

(52) U.S. Cl. 604/385.03; 604/385.06; 604/387; 604/389; 604/385.01

(57) ABSTRACT

A disposable, integrated diaper product is provided for use with a human. The product comprises a diaper assembly, a pair of opposing lateral tabs, a fastening section, a premoistened textile component, and a liquid impermeable component. The liquid impermeable component can releasably adhesively mate against the exterior component of the diaper assembly. The liquid impermeable sheet element can be disposed along a substantially straight line intermediate the opposing lateral tabs in a waist section of the exterior component in order to prevent interference with the attachment of the opposing lateral tabs in the fastening section.

### PRIORITY INFORMATION

This application is a non-provisional of U.S. Patent Application 60/896,856, filed Mar. 23, 2007, the entire content of which is hereby incorporated by reference herein.

### BACKGROUND OF THE INVENTION

1. Field of the Invention

The present inventions relate to a unique integrated product that can combine various complimentary products to allow easy access and quick application of a complimenting product when using a complimented product. In particular, various embodiments provide for a unique integrated sanitary product that can combine a cleansing product with a carrier product to allow easy access and quick application of the cleansing product when using the carrier product.

2. Description of the Related Art

Hygienic products, including diapers and female sanitary products, are frequently used with other cleansing agents, such as washcloths, wipes, and the like, in order to facilitate proper hygiene and cleanliness. In some embodiments of the present inventions, the unique integrated sanitary product can combine these products to allow easy access and quick application of the wipe product when changing the hygienic product. For example, in one application, when changing a baby's diaper, the caregiver can simply remove the wipe from its protective package on the outside of the diaper and use it to properly clean the baby before putting a new diaper on the baby. Once used, the wipe can be folded into and/or discarded with the used diaper.

### SUMMARY OF THE INVENTION

A comprehensive discussion is not provided of the various other applications in which embodiments of the present inventions can be used; however, it is contemplated that the illustrative figures and embodiments provided can enable one of skill in the art to apply these teachings to a variety of products, for hygienic and other uses. Thus, although hygienic uses in diapers and female sanitary products are disclosed as examples, it is contemplated that various other embodiments are generally disclosed for uses with products that require a user to apply a certain procedure, material, or operation to an object, workpiece, or to the user herself after use. For example, containers for finger foods, brake pads, toner cartridges, and other potentially messy items can profitably incorporate embodiments disclosed herein.

In accordance with an embodiment, a disposable, integrated diaper product is provided for use with a human. The product comprises a diaper assembly, a pair of opposing lateral tabs, a fastening section, a premoistened textile component, and a liquid impermeable sheet element.

The diaper assembly can have an absorbent interior component and an exterior component. The absorbent interior component of the diaper assembly can be coupled to the exterior component. The exterior component can comprise a generally impermeable material. The exterior component of the diaper assembly can define a first end and a second end. The exterior component can comprise opposing first and second waist sections being disposed at the respective ones of the first and second ends of the exterior component.

The pair of opposing lateral tabs can be disposed at the first end of the exterior component of the diaper assembly. The opposing lateral tabs can extend in generally opposite directions along a substantially straight line being oriented generally transverse relative to a longitudinal axis of the diaper assembly. The fastening section can be disposed at the second end of the exterior component. The fastening section of the exterior component can be configured such that the pair of opposing lateral tabs are attachable thereto for securing the diaper assembly onto the human with the absorbent interior component being located interiorly relative to the exterior component.

The premoistened textile component can be made from one of a woven and a nonwoven fabric. Further, the liquid impermeable sheet element can define an adhesive interior surface and an exterior surface. The liquid impermeable sheet element can be configured to adhesively mate against the exterior component of the diaper assembly with the adhesive interior surface contacting the exterior component of the diaper assembly. The liquid impermeable sheet element can be releasably sealed against the exterior component with the premoistened textile component captured against the diaper assembly for maintaining the moisture content of the premoistened textile component.

In such an embodiment, the liquid impermeable sheet element can be disposed along the substantially straight line intermediate the opposing lateral tabs in the first waist section of the exterior component. The liquid impermeable sheet element can thereby be disposed on a region of the exterior component wherein the liquid permeable sheet element and the premoistened textile component do not interfere with the placement and attachment of the opposing lateral tabs in the fastening section of the exterior component.

In addition, the liquid impermeable sheet element can further comprise a tab portion for pulling and separating the liquid impermeable sheet element from the exterior component. Further, the diaper assembly can define a midline being oriented orthogonally relative to the longitudinal axis of the diaper assembly. The midline can be disposed approximately equidistant between the first and second ends of the diaper assembly to divide the exterior component into first and second regions. The first region can be disposed adjacent to the first end and the second region can be disposed adjacent to the second end. In this manner, the liquid permeable sheet element can be attached to the exterior component of the diaper assembly only in the first region.

In accordance with another embodiment, the liquid impermeable sheet element can be releasably sealed against the exterior component with the premoistened textile component captured intermediate the liquid impermeable sheet element and the exterior component of the diaper assembly for maintaining the moisture content of the premoistened textile component. The liquid impermeable sheet element can provide a fluid-tight seal with the exterior component of the diaper assembly. The premoistened textile component can be in direct contact with the exterior component of the diaper assembly.

In some embodiments, the liquid impermeable sheet can be a single layer of material. Further, the liquid impermeable sheet element can comprise a pouch defining an interior area wherein the premoistened textile component can be disposed. The liquid impermeable sheet element can further comprise a Velcro surface for attaching to the outer surface of the exterior component of the diaper assembly. Further, the liquid impermeable sheet element can comprise packaging for the premoistened textile component.

In accordance with an embodiment, a disposable, integrated diaper product is provided for use with a human. The product comprises a diaper assembly, a pair of opposing lateral tabs, a fastening section, a premoistened textile component, and a liquid impermeable pouch component.

The diaper assembly can have an absorbent interior component and an exterior component. The absorbent interior component of the diaper assembly can be coupled to the exterior component. The exterior component can comprise a generally impermeable material. The exterior component of the diaper assembly can define a first end and a second end. The exterior component can comprise opposing first and second waist sections being disposed at the respective ones of the first and second ends of the exterior component.

The pair of opposing lateral tabs can be disposed at the first end of the exterior component of the diaper assembly. The opposing lateral tabs can extend in generally opposite directions along a substantially straight line being oriented generally transverse relative to a longitudinal axis of the diaper assembly. The fastening section can be disposed at the second end of the exterior component. The fastening section of the exterior component can be configured such that the pair of opposing lateral tabs are attachable thereto for securing the diaper assembly onto the human with the absorbent interior component being located interiorly relative to the exterior component.

The premoistened textile component can be made from one of a woven and a nonwoven fabric. The liquid impermeable pouch component can define first and second surfaces. The liquid impermeable pouch component can fasten the premoistened textile component against the exterior component of the diaper assembly. The liquid impermeable pouch component can be configured to releasably adhesively mate against the exterior component of the diaper assembly. Further, the liquid impermeable pouch component can be disposed along the substantially straight line intermediate the opposing lateral tabs in the first waist section of the exterior component. The liquid impermeable pouch component can thereby not interfering with the placement and attachment of the opposing lateral tabs in the fastening section of the exterior component.

The tab portion can be coupled to the liquid impermeable pouch component for pulling and separating the liquid impermeable pouch component from the exterior component.

In some embodiments, the diaper assembly can defines a midline being oriented orthogonally relative to the longitudinal axis of the diaper assembly. The midline can be disposed approximately equidistant between the first and second ends of the diaper assembly to divide the exterior component into first and second regions. The first region can be disposed adjacent to the first end and the second region can be disposed adjacent to the second end. The liquid permeable pouch component can be attached to the exterior component of the diaper assembly only in the first region.

Further, the liquid impermeable pouch component can be releasably sealed against the exterior component with the premoistened textile component captured intermediate the liquid impermeable pouch component and the exterior component of the diaper assembly for maintaining the moisture content of the premoistened textile component. The liquid impermeable pouch component can provide a fluid-tight seal with the exterior component of the diaper assembly. The premoistened textile component can be in direct contact with the exterior component of the diaper assembly.

In yet other embodiments, the liquid impermeable pouch component can be a single layer of material. The liquid impermeable pouch component can comprise top and bottom layers and define an interior area between the top and bottom layers. In such an embodiment, the premoistened textile component can be enveloped in the interior area of the liquid impermeable pouch component. The liquid impermeable pouch component can further comprise a Velcro surface for attaching to the outer surface of the exterior component of the diaper assembly. The liquid impermeable pouch component can comprise packaging for the premoistened textile component.

### BRIEF DESCRIPTION OF THE DRAWINGS

The abovementioned and other features of the inventions disclosed herein are described below with reference to the drawings of the preferred embodiments. The illustrated embodiments are intended to illustrate, but not to limit the inventions. The drawings contain the following figures:

FIG. 1 is a side view of an embodiment of an integrated product.

FIG. 2 is a perspective view of the product shown in FIG. 1.

FIG. 3 is a perspective view of another embodiment of an integrated product having a wipe product with a removable edge for accessing a cleansing agent disposed in the wipe product.

FIG. 4 is a perspective view of the product of FIG. 3 wherein the edge of the wipe product has been removed.

FIG. 5 is a side view of system a unitary products, according to yet another embodiment.

FIG. 6 is a perspective view of another embodiment of the integrated product.

FIG. 7A is a cross-sectional side view of a sheet element or pouch component, in accordance with an embodiment.

FIG. 7B is a cross-sectional side view of the sheet element or pouch component, in accordance with another embodiment.

FIG. 8 is a side vie w of an embodiment of the integrated product in an as-used configuration.

### DETAILED DESCRIPTION OF THE PREFERRED EMBODIMENT

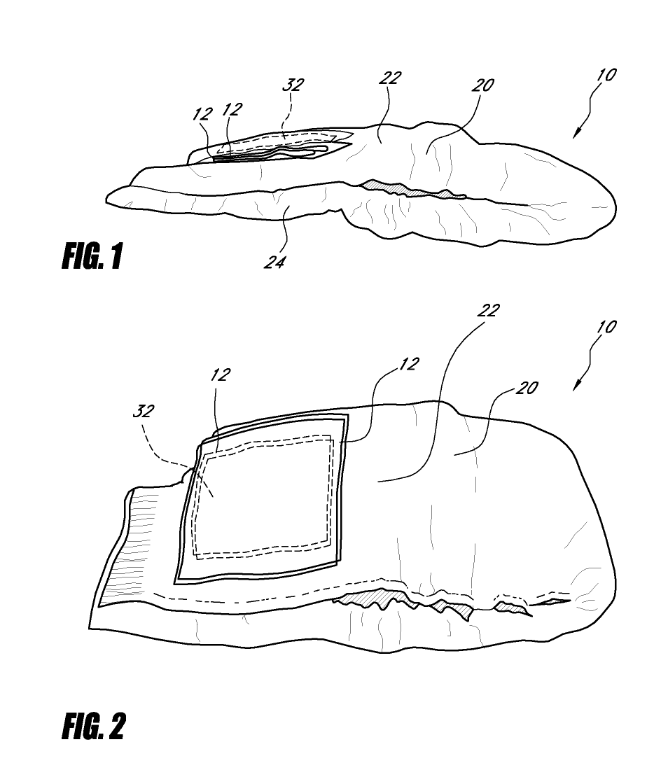

Referring now to the embodiments disclosed in the drawings, FIG. 1 is an embodiment illustrating an integrated sanitary product 10 in which a wipe product 12 can be attached to a hygienic product 20. In some embodiments, the wipe product 12 can be any variety of sanitary napkins, cloths, liquids, antibacterial gel, or other materials. For example, the wipe product 12 can be a disposal cloth that is pre-moistened with a solution of gentle cleansing ingredients. The wipe product 12 can include single or multiple cloths. The hygienic product 20 can be any variety of hygienic products, including diapers, feminine hygiene products, feminine sanitary products, such as tampons and the like, bandages, and any other product that may be applied to the skin in order to aid in maintaining personal cleanliness.

As shown in FIGS. 1-5, the wipe product 12 can be attached to the hygienic product 20 on an outer surface 22 thereof. The wipe product 12 can include a wrapper 30 that at least partially encompasses a sanitary napkin 32, which can be a pre-moistened disposal cloth, as described above. For example, in an embodiment wherein the wrapper 30 completely surrounds the sanitary napkin 32, such that the sanitary napkin 32 is sealed within the wrapper 30, the wrapper 30 of the wipe product 12 can be directly attached to the outer surface 22 of the hygienic product 20. In some embodiments, the wipe product 12 can be permanently attached to the outer surface 22, thus requiring that the wipe product 12 be opened by tearing, for example, to allow removal of the sanitary napkin 32.

In other embodiments, the wrapper 30 of the wipe product 12 can be releasably attached to the outer surface 22 by means of an adhesive. In yet other embodiments, the wipe product 12 can be configured to fit within a holding means disposed on the outer surface 22 of the hygienic product 20. The holding means (not shown) can include an external pocket, strap, or other physical feature of the hygienic product 20 that can be used to retain the wipe product 12 joined to the hygienic product 20.

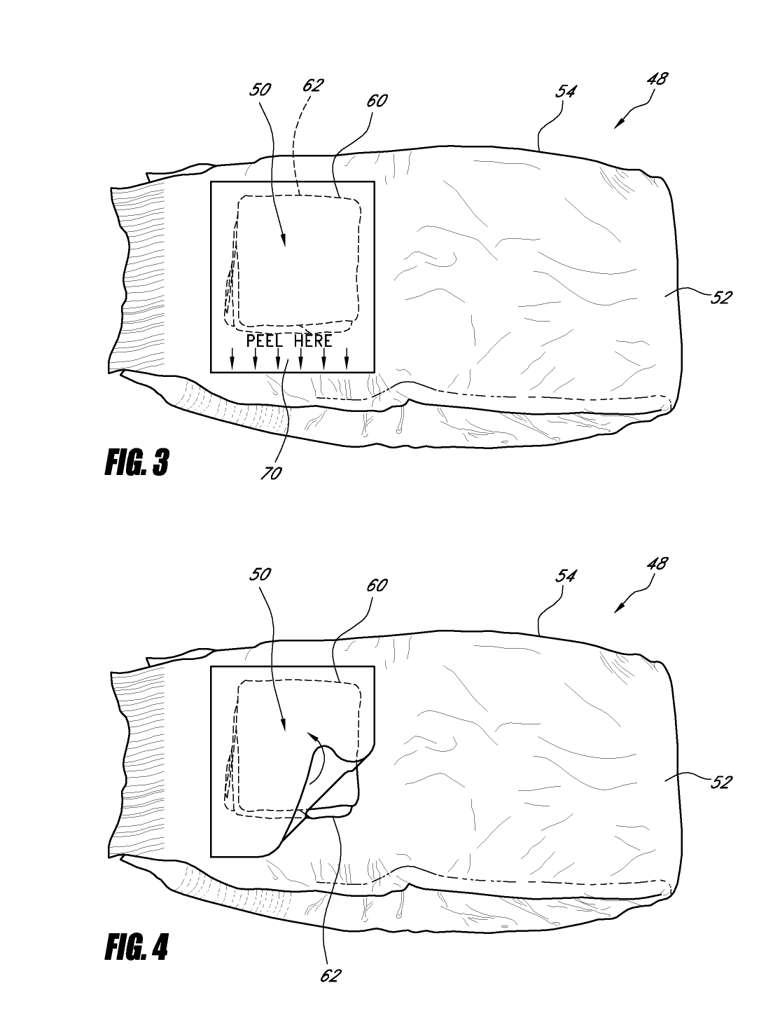

In accordance with an implementation of such embodiments, it is also contemplated that multiple wipe products 12 can be coupled together and joined to the outer surface 22 of the hygienic product 20. For example, as illustrated in FIG. 2, two wipe products 12 can be adhesively attached to an exterior surface 22 of a diaper 24.

The attachment of the wipe product 12 to the hygienic product 20 can be facilitated by using any variety of fastening means, such as glue, double sided tape, other sticky materials, Velcro, and the like. In an embodiment, the wrapper 30 of the wipe product 12 can incorporate a Velcro surface that allows the wipe product 12 to be attached to a woven front section of the exterior surface of the hygienic product 20. Such an embodiment can allow easy removal and replacement of the wipe product 12 to form an embodiment of an integrated system of replaceable wipe products utilizable with hygienic products. In such a system embodiment, the wipe product 12 can utilize various adhesive properties in attaching to the outer surface 22 of the hygienic product 20. Other various embodiments and structures are also contemplated and are encompassed in the present disclosure.

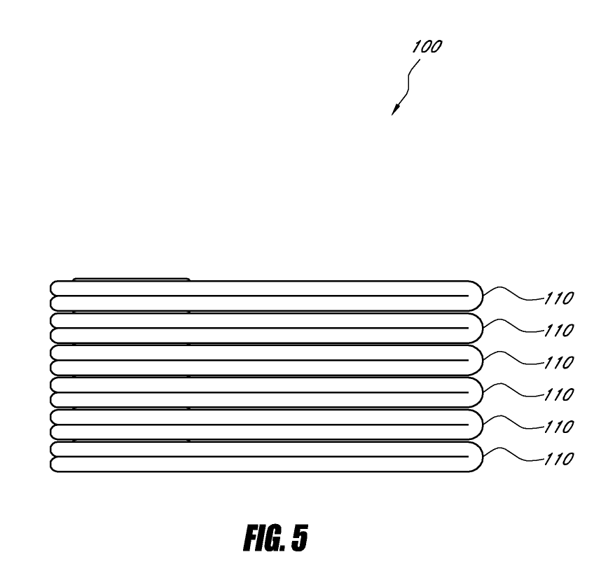

Referring now to FIGS. 3 and 4, another embodiment of an integrated product 48 is illustrated in a perspective view. As shown in FIG. 3, a wipe product 50 can be attached to an external surface 52 of a hygienic product 54. However, in this embodiment, a wrapper 60 of the wipe product 50 is configured to only partially envelope a sanitary napkin 62. Thus, the sanitary napkin 62 is bounded on at least one of its sides by the external surface 52 of the hygienic product 54. Accordingly, where the sanitary napkin 62 is moistened or incorporates a liquid or gel product, the external surface 52 of the hygienic product 54 is preferably impervious to moisture. Thus, the illustrated embodiment of FIG. 3 shows that the sanitary napkin 62 could be placed against the external surface 52 of a diaper, and that the wrapper 60 could be placed on top of the sanitary napkin 62 and can provide a seal with the external surface 52 to seal in moisture and ensure cleanliness of the napkin 62. In such an embodiment, the wrapper 60 can be a press-and-seal material to facilitate the manufacture of the product 10.

As also illustrated in FIGS. 3-4, the wrapper 60 could have at least one removable edge 70 that could be peeled away from the external surface 52 in order to access the sanitary napkin 62. FIG. 4 shows that the removable edge 70 has been peeled away to expose the sanitary napkin 62 disposed between the wrapper 60 and the hygienic product 54. Such an embodiment would be advantageous because it can provide a waterproof seal to maintain the moisture content of a pre-moistened sanitary napkin. However, most importantly, the cleanliness of the sanitary napkin 62 can be maintained.

The embodiment shown in FIGS. 3 and 4 may provide various manufacturing advantages and enable the user to quickly access a sanitary napkin when changing the hygienic product. In this regard, each of the embodiments disclosed herein can make the cleaning process much easier for the user of the integrated product. For example, many parents have frequently encountered the unfortunate situation where they must urgently change a diaper, such as in a blowout, but cannot locate baby wipes needed to clean the baby and to change the diaper. Instead of having to spend extra time trying to find misplaced baby wipes, the parent can simply use the baby wipe(s) that is integrated into the unitary hygienic product itself. In this regard, parents are provided with all of the tools that they need to quickly change the baby's diaper, resulting in a much faster process and increased happiness for the parent and the baby.

Indeed, embodiments of the inventions disclosed herein can effectively eliminate the need for carrying or purchasing several types of supplies. This can be particularly advantageous where parents are running errands and need to take the baby along with them. Instead of having to take a large container of pre-moistened baby wipes, the parents need only take a couple of diapers that are configured according to embodiments disclosed herein. It is also advantageous for the on-the-go lifestyle of many people; often cleaning agents are not immediately available before eating finger foods, after changing toner in the copier, and myriad other situations that could be made easier with quick access to cleansing agents. Numerous other advantages and features are apparent to one of skill in the art.

As mentioned above, it is also contemplated that the embodiments disclosed herein can be used with any variety of hygienic products, including feminine products. In this regard, the wipe product can be configured in a variety of sizes and shapes so as to facilitate attachment with any variety of hygienic products. For example, the wipe product can be more elongate than square in shape so as to compliment the shape of a tampon.

Further, as noted in the above-disclosed embodiments, the configuration of the wipe product, and in particular, the configuration of the wrapper, can be modified to only partially encompass or envelope the sanitary napkin disposed therein and can thus utilize an external surface of the hygienic product as a backing for the wipe product, thus reducing the bulk and size of products incorporating one of the embodiments disclosed herein.

In accordance with another embodiment, it is contemplated that the wipe product can include an easy peel mechanism for facilitating the removal of the sanitary napkin from the wipe product. In this regard, it is contemplated that the wrapper of the wipe product can be configured to include a tab portion. In such an embodiment, the tab portion can be directly attached and/or stitched to an edge of the sanitary napkin disposed within the wipe product. The tab portion can be exposed on an outer surface of the wipe product so that the tab portion can be pulled by the user.

In use, the tab portion of the wipe product could be peeled away from the wipe product by the user, thus opening the wrapper and, because the tab portion is attached to the sanitary napkin, continued pulling of the tab portion can effectively remove the sanitary napkin from within the wrapper of the wipe product. Thus, the sanitary napkin can be removed from within the wipe product in a very simple one-step peeling/pulling process. This feature can be particularly advantageous because it can enable the user to quickly access the sanitary napkin and can therefore greatly speed up the cleaning process. In addition, because the removable tab portion would remain attached to the sanitary napkin, the user need not worry about picking up small wrappers that would otherwise need to be collected and discarded. Thus, the user is left with only two items to discard: the napkin, with the tab portion being attached thereto, and the hygienic product, to which the wrapper of the wipe product remains attached.

Additionally, as mentioned above, it is contemplated that some of the embodiments can include a plurality of sanitary napkins disposed within the wrapper of the wipe product. Alternatively, some of the embodiments can be configured to include multiple wipe products disposed on the exterior of the hygienic product, which is illustrated in FIGS. 1-2. Thus, when needed, multiple sanitary napkins can be easily accessed by the user and used at their discretion.

In accordance with another embodiment, FIG. 5 illustrates a system 100 of integrated hygienic products 110. The system 100 can include a plurality of products 110 that incorporate the combination hygienic product and wipe products, as described above. The system 100 can be packaged as a single unit and provided to the user, thus allowing the user to have a plurality of individual hygienic products, each having an attached wipe product.

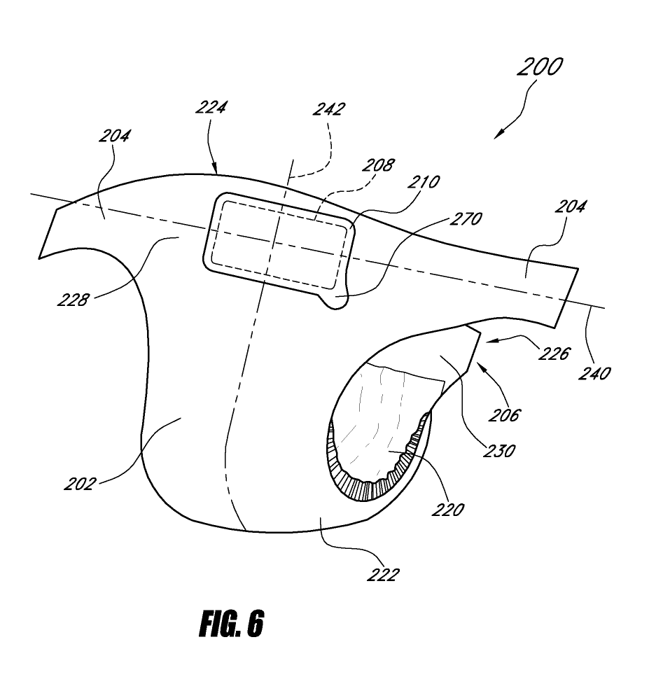

Referring now to FIG. 6, another embodiment of a disposable, integrated diaper product 200 is shown. The product 200 can comprise a diaper assembly 202, a pair of opposing lateral tabs 204, a fastening section 206, a textile component 208, and a sheet element or pouch component 210.

The diaper assembly 202 can have an absorbent interior component 220 and an exterior component 222. The absorbent interior component 220 of the diaper assembly 202 can be coupled to the exterior component 222 as done in the art. The exterior component 222 can comprise a generally impermeable material, such as a plastic or other suitable material. The exterior component 222 of the diaper assembly 202 can define a first end 224 and a second end 226. The exterior component 222 can comprise opposing first and second waist sections 228, 230 being disposed at the respective ones of the first and second ends 224, 226 of the exterior component 222.

The pair of opposing lateral tabs 204 can be disposed at the first end 224 of the exterior component 222 of the diaper assembly 202. The opposing lateral tabs 204 can extend in generally opposite directions along a substantially straight line 240 being oriented generally transverse relative to a longitudinal axis 242 of the diaper assembly 202. The fastening section 206 can be disposed at the second end 226 of the exterior component 202. The fastening section 206 of the exterior component 222 can be configured such that the pair of opposing lateral tabs 206 are attachable thereto for securing the diaper assembly 202 onto the human with the absorbent interior 220 component being located interiorly relative to the exterior component 222.

The textile component 208 can be made from a woven or nonwoven fabric. The component 208 preferably is premoistened with an appropriate liquid for cleansing skin, for example. Further, the sheet element or pouch component 210 can be liquid impermeable in some embodiments.

In accordance with some embodiments, the sheet element 210 can define an adhesive interior surface 250 and an exterior surface 252. The sheet element 210 can adhesively mate against the exterior component 222 of the diaper assembly 202 with the adhesive interior surface 250 contacting the exterior component 222 of the diaper assembly 202. The adhesive coupling can be achieved using any variety of safe and non-permanent adhesives. Preferably, a releasable adhesive can be used to provide a tacky surface for the interior surface 250 to thereby allow the interior surface 250 to sealingly mate with the exterior component 222. Thus, an embodiment, a liquid impermeable sheet element can be releasably sealed against the exterior component 222 with a premoistened textile component captured against the diaper assembly for maintaining the moisture content of the premoistened textile component.

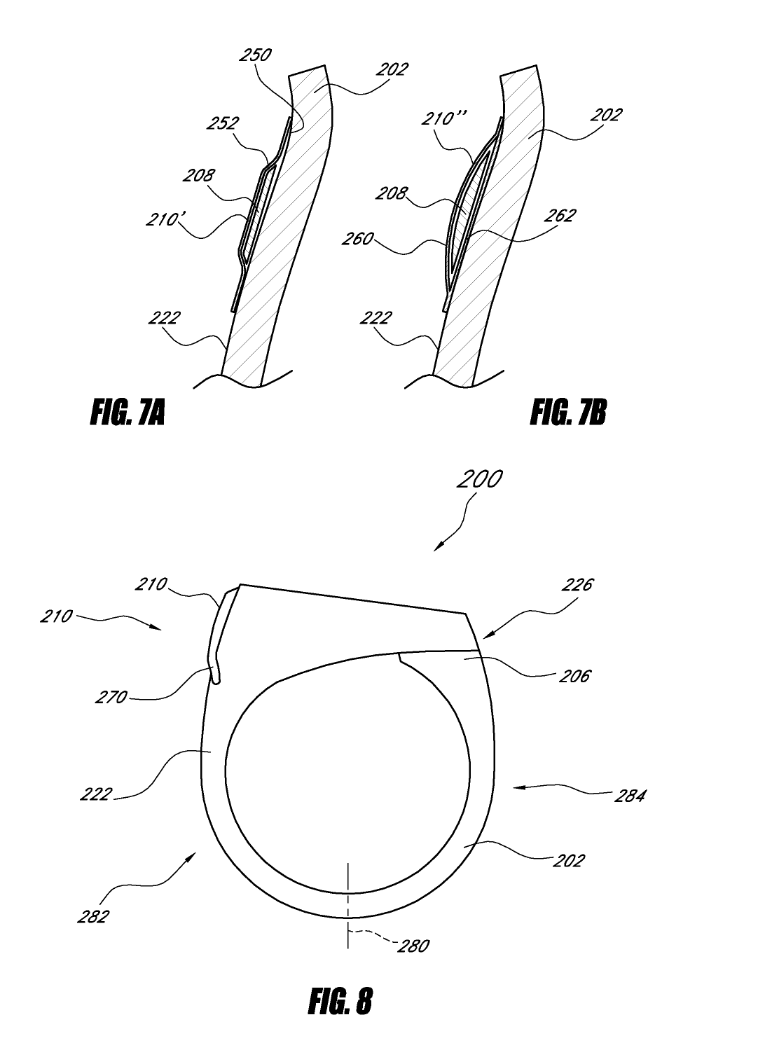

For example, FIG. 7A is a cross-sectional view of an embodiment of a sheet element, diaper assembly, and textile component. As shown in FIG. 7A, a liquid impermeable sheet element 210′ can be releasably sealed against the exterior component 222 with a premoistened textile component 208 captured intermediate the sheet element 210 and the exterior component 222 of the diaper assembly 200 for maintaining the moisture content of the premoistened textile component 208. In this regard, the liquid impermeable sheet element 210′ can provide a fluid-tight seal with the exterior component 222 of the diaper assembly 200.

Further, in some embodiments, a premoistened textile component can actually be in direct contact with the exterior component 222 of the diaper assembly 200. In such an embodiment, both the exterior component 222 and the sheet element 210 should be liquid impermeable. In such an embodiment, the sheet element 210 can be a single layer of material. However, the sheet element 210 can also be configured to include one or more layers of material, as described further below.

In yet other embodiments, such as that illustrated in the cross-sectional view of FIG. 7B, the sheet element 210″ can be similar to a pouch component and can define first and second layers or surfaces 260, 262. The sheet element or pouch component 210″ can fasten the premoistened textile component 208 against the exterior component 222 of the diaper assembly 202. The sheet element or pouch component 210″ can be a liquid impermeable sheet element or pouch component and can be configured to releasably adhesively mate against the exterior component 222 of the diaper assembly 202.

Further, the second layer or surface 262 of the liquid impermeable sheet element 210″ can comprise a Velcro surface for attaching to the outer surface of the exterior component 222 of the diaper assembly 202. Other suitable adhesive materials can also be used. In such embodiments, the liquid impermeable sheet element or pouch component 210″ can also comprise packaging for the premoistened textile component. For example, the sheet element or pouch component 210″ can be comparable in structure to a Wet Nap®.

Further, the sheet element or pouch component 210 can be disposed along the substantially straight line 240 intermediate the opposing lateral tabs 204 in the first waist section 228 of the exterior component 222. This arrangement can beneficially allow the sheet element or pouch component 210 to be placed on the diaper product 200 in such a way that the sheet element or pouch component 210 does not interfere with the compressibility or flexibility of the diaper assembly 202.

For example, walking and crawling tend to require flexibility and compressibility of a diaper in order to allow the full range of leg motion in an infant or toddler. By placing the sheet element or pouch component along the straight line 240, the sheet element or pouch component can be carried on the diaper without constricting or interfering with leg movement. Further, this positioning of the sheet element or pouch component also beneficially provides that the sheet element or pouch component will not be repeatedly bended, stretched, or otherwise jostled during use. This feature is generally illustrated in FIG. 8.

Accordingly, embodiments of the present inventions allow the sheet element or pouch component to maintain a superior seal for ensuring that the moisture content of the textile component is preserved. Furthermore, this unique placement of the sheet element or pouch component along the waist section intermediate the opposing lateral tabs can also tend to ensure that the sheet element or pouch component is easily accessible for a caregiver when attempting to change the diaper.

Furthermore, the placement of the sheet element or pouch component as discussed above can also beneficially allow the sheet element or pouch component not to interfere with the attachment of the opposing lateral tabs in the fastening section of the exterior component. Thus, the diaper can be firmly and securely placed on the child while ensuring that the sheet element or pouch component is also protected and maintained for use.

In accordance with some embodiments, the sheet element or pouch component 210 can further comprise a tab portion 270 as shown in FIGS. 6 and 8. The tab portion 270 can extend from the sheet element or pouch component 210 and allow the sheet element or pouch component 210 to be pulled and separated from the exterior component 222. In some embodiments, at least a portion of the tab portion 270 may not be adhesively attached to the exterior component 222 of the diaper assembly 202.

FIG. 8 shows an embodiment of the diaper product 200 in an as-worn configuration with the opposing lateral tabs 204 being attached to the fastening section 206. The diaper assembly 202 can define a midline 280 being oriented orthogonally relative to the longitudinal axis of the diaper assembly, as illustrated in FIG. 8. The midline 280 can be disposed approximately equidistant between the first and second ends 226, 228 of the diaper assembly 202 to divide the exterior component 222 into first and second regions 282, 284. The first region 282 can be disposed adjacent to the first end 228 and the second region 284 can be disposed adjacent to the second end 226. As discussed above, the sheet element or pouch component 210 can be attached to the exterior component 222 of the diaper assembly 202 only in the first region 282. Thus, any interference with the opposing lateral tabs 204 can be avoided and the sheet element or pouch component 210 can be situated in a relatively stable zone when the diaper is worn by the child.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

What is claimed is:

1. A disposable, integrated diaper product for use with a human, the product comprising:
a diaper assembly having an absorbent interior component and an exterior component, the absorbent interior component of the diaper assembly being coupled to the exterior component, the exterior component comprising a generally impermeable material, the exterior component of the diaper assembly defining a first end and a second end, the exterior component comprising opposing first and second waist sections being disposed at the respective ones of the first and second ends of the exterior component;
a pair of opposing lateral tabs disposed at the first end of the exterior component of the diaper assembly, the opposing lateral tabs extending in generally opposite directions along a substantially straight line being oriented generally transverse relative to a longitudinal axis of the diaper assembly;
a fastening section disposed at the second end of the exterior component, the fastening section of the exterior component being configured such that the pair of opposing lateral tabs are attachable thereto for securing the diaper assembly onto the human with the absorbent interior component being located interiorly relative to the exterior component;
a premoistened textile component being made from one of a woven and a nonwoven fabric; and
a liquid impermeable sheet element defining an adhesive interior surface and an exterior surface, the liquid impermeable sheet element being configured to adhesively mate against the exterior component of the diaper assembly with the adhesive interior surface contacting the exterior component of the diaper assembly, the liquid impermeable sheet element being releasably sealed against the exterior component with the premoistened textile component captured against the diaper assembly for maintaining the moisture content of the premoistened textile component, wherein the liquid impermeable sheet element is disposed along the substantially straight line intermediate the opposing lateral tabs in the first waist section of the exterior component, the liquid impermeable sheet element thereby being disposed on a region of the exterior component wherein the liquid permeable sheet element and the premoistened textile component do not interfere with the placement and attachment of the opposing lateral tabs in the fastening section of the exterior component.

2. The product of claim 1, wherein the liquid impermeable sheet element further comprises a tab portion for pulling and separating the liquid impermeable sheet element from the exterior component.

3. The product of claim 1, wherein the diaper assembly defines a midline being oriented orthogonally relative to the longitudinal axis of the diaper assembly, the midline being disposed approximately equidistant between the first and second ends of the diaper assembly to divide the exterior component into first and second regions, the first region being disposed adjacent to the first end and the second region being disposed adjacent to the second end, wherein the liquid permeable sheet element is attached to the exterior component of the diaper assembly only in the first region.

4. The product of claim 1, wherein the liquid impermeable sheet element is releasably sealed against the exterior component with the premoistened textile component captured intermediate the liquid impermeable sheet element and the exterior component of the diaper assembly for maintaining the moisture content of the premoistened textile component.

5. The product of claim 4, wherein the liquid impermeable sheet element provides a fluid-tight seal with the exterior component of the diaper assembly.

6. The product of claim 5, wherein the premoistened textile component is in direct contact with the exterior component of the diaper assembly.

7. The product of claim 1, wherein the liquid impermeable sheet is a single layer of material.

8. The product of claim 1, wherein the liquid impermeable sheet element comprises a pouch defining an interior area wherein the premoistened textile component can be disposed.

9. The product of claim 7, wherein the liquid impermeable sheet element further comprises a Velcro surface for attaching to the outer surface of the exterior component of the diaper assembly.

10. The product of claim 1, wherein the liquid impermeable sheet element comprises packaging for the premoistened textile component.

11. A disposable, integrated diaper product for use with a human, the product comprising:
a diaper assembly having an absorbent interior component and an exterior component, the absorbent interior component of the diaper assembly being coupled to the exterior component, the exterior component comprising a generally impermeable material, the exterior component of the diaper assembly defining a first end and a second end, the exterior component comprising opposing first and second waist sections being disposed at the respective ones of the first and second ends of the exterior component;
a pair of opposing lateral tabs disposed at the first end of the exterior component of the diaper assembly, the opposing lateral tabs extending in generally opposite directions along a substantially straight line being oriented generally transverse relative to a longitudinal axis of the diaper assembly;
a fastening section disposed at the second end of the exterior component, the fastening section of the exterior component being configured such that the pair of opposing lateral tabs are attachable thereto for securing the diaper assembly onto the human with the absorbent interior component being located interiorly relative to the exterior component;
a premoistened textile component being made from one of a woven and a nonwoven fabric;
a liquid impermeable pouch component defining first and second surfaces, the liquid impermeable pouch component fastening the premoistened textile component against the exterior component of the diaper assembly, the liquid impermeable pouch component being configured to releasably adhesively mate against the exterior component of the diaper assembly, wherein the liquid impermeable pouch component is disposed along the substantially straight line intermediate the opposing lateral tabs in the first waist section of the exterior component, the liquid impermeable pouch component thereby not interfering with the placement and attachment of the opposing lateral tabs in the fastening section of the exterior component; and
a tab portion being coupled to the liquid impermeable pouch component for pulling and separating the liquid impermeable pouch component from the exterior component.

12. The product of claim 11, wherein the diaper assembly defines a midline being oriented orthogonally relative to the longitudinal axis of the diaper assembly, the midline being disposed approximately equidistant between the first and second ends of the diaper assembly to divide the exterior component into first and second regions, the first region being disposed adjacent to the first end and the second region being disposed adjacent to the second end, wherein the liquid permeable pouch component is attached to the exterior component of the diaper assembly only in the first region.

13. The product of claim 11, wherein the liquid impermeable pouch component is releasably sealed against the exterior component with the premoistened textile component captured intermediate the liquid impermeable pouch component and the exterior component of the diaper assembly for maintaining the moisture content of the premoistened textile component.

14. The product of claim 13, wherein the liquid impermeable pouch component provides a fluid-tight seal with the exterior component of the diaper assembly.

15. The product of claim 14, wherein the premoistened textile component is in direct contact with the exterior component of the diaper assembly.

16. The product of claim 11, wherein the liquid impermeable pouch component is a single layer of material.

17. The product of claim 11, wherein the liquid impermeable pouch component comprises top and bottom layers and defines an interior area between the top and bottom layers, wherein the premoistened textile component is enveloped in the interior area of the liquid impermeable pouch component.

18. The product of claim 17, wherein the liquid impermeable pouch component further comprises a Velcro surface for attaching to the outer surface of the exterior component of the diaper assembly.

19. The product of claim 1, wherein the liquid impermeable pouch component comprises packaging for the premoistened textile component.
